(19) United States
(12) Patent Application Publication
(10) Pub. No.: US 2002/0127192 A1
(43) Pub. Date: Sep. 12, 2002

(54) Lipstick with translucent or transparent region

(76) Inventors: John Murphy, Fayetteville, TN (US); Angie Sanders, Lewisburg, TN (US)

Correspondence Address: Marshall, O'Toole, Gerstein, Murray & Borun, 6300 Sears Tower, 233 South Wacker Drive, Chicago, IL, 60606-6402, US

(21) Appl. No.: 09/754,453

(22) Filed: Jan. 4, 2001

(51) Int. Cl. A61K 7/025

(52) U.S. Cl. 424/64

(57) ABSTRACT

A lipstick including a plurality of regions wherein at least one of the regions is translucent or transparent is described.

### BACKGROUND OF THE INVENTION

1. Field of the Invention

The invention relates generally to cosmetic articles for use on the lips and, more specifically, lipsticks having a translucent or transparent region.

2. Brief Description of Related Technology

Cosmetic articles for direct application on the lips include lipsticks, lip liners, and lip pencils. Cosmetic compositions for use in these articles are sometimes referred to by the same name (e.g., a lip liner article includes a lip liner composition), but often a cosmetic article can be made up of various cosmetic compositions singly or in combination (e.g., a lipstick can be made of lip rouge, lip balm, lip gloss, lip liner, and combinations thereof). The consistency of a cosmetic composition can vary from solid, to semi-solid, to liquid. The manipulation of the consistency of the composition allows for formation of the composition into various cosmetic articles. For example, lip gloss compositions can be liquids that are applied with an applicator, low-viscosity semi-solids stored in a pot and applied with a finger, and high-viscosity semi-solids or solids formed as a lipstick and applied directly by ruboff.

Most conventional lipsticks are produced in a “bullet” shape having a tapered cylindrical body and having a diameter at the bottom of about ½ inch. The upper part of the lipstick bullet can be provided with various shapes known in the industry, including fishtail (also known as fish fin), chisel cut, teardrop, contoured, blunt, oval, ogival (having a pointed arch), and beveled. The shaped upper portion of the lipstick also can be provided with a face. The bottom portion of the bullet is seated in a “cup” of the lipstick case. The “cup” can be lowered and raised in the lipstick case for storage (with a suitable cover) and application, respectively.

Traditional lipsticks have a substantially uniform composition throughout the article, but other arrangements are known in the art. For example, U.S. Pat. No. 3,279,999 (Oct. 18, 1966) discloses a self-shaping lipstick having regions made of compositions with different hardness; U.S. Pat. No. 3,479,429 (Nov. 18, 1969) discloses multi-colored lipsticks wherein differently colored masses are heterogeneously associated in the form of a unitary article; U.S. Pat. No. 4,291,018 (Sep. 22, 1981) discloses a lipstick having a construction of the core-sheath type including two different compositions arranged in core-sheath relationship; Japanese Patent Publication No. 61-112008 (May 30, 1986) discloses a multi-colored lipstick having two or three different compositions arranged in core-sheath and several side-by-side relationships; and U.S. Pat. No. 4,743,443 (May 10, 1988) discloses a tri-color or alternating bi-color lipstick wherein the different compositions are arranged in side-by-side relationship.

In practice, lip gloss products are typically applied over an application of lip rouge on the lips or, in some cases, both under and over an application of lip rouge on the lips. This practice requires the inconveniences of carrying at least two separate products and using two or three separate application steps. Moreover, it is often desirable to achieve blending of an undercoat of lip gloss with a layer of lip rouge, which is not readily achieved when the user must employ two products in serial fashion.

Lipsticks are not simply utilitarian items, but the appearance of the articles themselves is as important to the user as the appearance of the compositions on the lips of the user. Thus, various improvements in the presentation and appearance of lipsticks have been introduced, such as departures from the traditional cylindrical bullet shape (see U.S. Pat. No. 4,740,097 (Apr. 26, 1988), disclosing a heart-shaped lipstick), departures from the traditional solid-color appearance (see U.S. Pat. No. 3,479,429 (Nov. 18, 1969), disclosing lipsticks with marbleized or other patterns of color), the use of pearlescent pigments and glitter components, and surface treatments of lipstick molds to improve the appearance of molded lipstick products.

Accordingly, it would be desirable to provide a lipstick article having a high fashion appearance and which allows the user to blend lip gloss and lip rouge or color components onto the lips from a single article in a single application step.

### SUMMARY OF THE INVENTION

It is an objective of the invention to overcome one or more of the problems described above.

Accordingly, one aspect of the invention is a lipstick including a plurality of regions wherein at least one of the regions is translucent or transparent.

In a lipstick of the core-sheath type including two different compositions disposed in a core-sheath relationship along a longitudinal axis thereof, another aspect of the invention provides an improvement wherein a composition disposed as a sheath is translucent or transparent.

Yet another aspect of the invention is a lipstick including a plurality of substantially coextensive regions extending lengthwise of the lipstick wherein at least one of the regions is transparent ro translucent, a first region is surrounded by a second region, at least one axial end of the first region is exposed, and the first region includes a color component.

Further aspects and advantages of the invention may become apparent to those skilled in the art from a review of the following detailed description, taken in conjunction with the appended claims. While the invention is susceptible of embodiments in various forms, described hereinafter are specific embodiments of the invention with the understanding that the disclosure is illustrative, and is not intended to limit the invention to the specific embodiments described herein.

### BRIEF DESCRIPTION OF THE DRAWINGS

FIG. 1 is a broken-away perspective view of an embodiment of the invention wherein the lipstick is the core-sheath type.

FIG. 2 is a cross-sectional view of an embodiment of the invention wherein the lipstick is the core-sheath type.

### DETAILED DESCRIPTION OF THE INVENTION

The invention is directed to a lipstick having a plurality of regions wherein at least one of the regions is translucent or transparent. Examples of a lipstick having a plurality of regions include lipsticks of the core-sheath type and lipsticks wherein two or more different compositions are arranged in substantially coextensive side-by-side relationship extending lengthwise of said lipstick.

Various abbreviations used herein are defined as follows: “C” is degree centigrade; “atm” is atmosphere; “cm” is centimeter; “HLB” is hydrophobic-lipophilic balance; “Hg” is mercury; “mm” is millimeter; “nm” is nanometer; and “UV” is ultraviolet.

The terms “transparent” and “translucent” as used herein, unless otherwise specified, are intended to connote their usual dictionary definitions. Thus, a transparent substance, like glass, allows ready viewing of objects behind the substance. A translucent substance allows light to pass through, but causes the light to be so scattered that it is difficult or impossible to clearly identify objects behind the translucent substance. For example, a composition is transparent if the maximum transmittance of light of any wavelength in the range about 200 nm to about 800 nm through a sample 10 cm thick is at least about 5%. Similarly, for example, a composition is translucent if such light through the sample is between about 0.01% and about 5%. The term “opaque” means that the maximum transmittance of such light is below about 0.01%. Transmittance can be easily measured by placing a sample of the composition having the required thickness in the light path of a UV-VIS Spectrophotometer such as the Hewlett-Packard 8451A Diode Ray Spectrophotometer. The advantage of this method of assessing transparency is that it is highly sensitive to optical clarity while independent of color.

The phrase “ambient conditions” as used herein, unless otherwise specified, refers to surrounding conditions at about one atmosphere of pressure (1 atm), at about 50% relative humidity, and at about 25° C.

The phrase “essentially free” as used herein, unless otherwise specified, is defined as meaning that the component (e.g., a wax) is not intentionally added to the composition, but may be present in small amounts as a contaminant or as a by-product in an ingredient of the composition.

The term “liquid” as used herein, unless otherwise specified, refers to a material that is substantially amorphous (noncrystalline) at ambient conditions.

The term “volatile” as used herein, unless otherwise specified, refers to a material that is liquid at ambient conditions and that has a vapor pressure of at least about 2 mm of mercury (mmHg) at 25° C. The term “nonvolatile” as used herein, unless otherwise specified, refers to those materials which are not volatile as that term is defined herein.

The various regions of a lipstick of the invention can be made up of any composition suitable for use on the lips including, but not limited to, lip rouges, lip glosses, lip balms, lip liners, and mixtures thereof. The primary components of typical compositions suitable for use on the lips are a structure-providing component, such as a wax (e.g., carnauba), and an emollient, such as an oil (e.g., castor), an ester, an oily alcohol (e.g., octyl dodecanol), an organically modified silicone (e.g., phenyltrimethicone alkyl dimethicones), and a triglyceride. Generally, the viscosity of the composition, due to the selected structural component, emollient, and optional ingredients, determines whether the composition will be transferrable to the lips of the user by rubbing off. When a color component, such as an organic pigment (e.g., D & C Red No. 7) is used, typically a solvent, such as an oil (e.g., castor oil) is used to wet out the pigment.

Compositions used to form regions of a lipstick of the invention preferably are made from ingredients that have been approved, at a minimum, for incidental ingestion. Examples of suitable approved ingredients are those “Generally Recognized As Safe” (GRAS) by the U.S. Food and Drug Administration (FDA), and those listed in the Food Chemicals Codex, the International Codex Alimentarius, the United States Pharmacopoeia (USP), the European Pharmacopoeia (EP), the Japanese Pharmacopoeia (JP) and the British Pharmacopoeia (BP).

Suitable structure-providing components useful in the invention include, but are not limited to, waxes, cetyl alcohol, stearyl alcohol, hydrogenated cottonseed oil, hydrogenated castor oil, hydrogenated palm oil, and mixtures thereof. Another suitable structure-providing component based on a polyamide resin is described fully below in conjunction with a preferred composition useful to form a translucent or transparent region in a lipstick of the invention.

Waxes are lower-melting organic mixtures or compounds of high molecular weight, solid at ambient conditions, and generally are similar in composition to fats and oils except that they contain no glycerides. They can be hydrocarbons or esters of fatty acids and alcohols. Waxes generally can be categorized as animal waxes, vegetable waxes, mineral waxes, natural waxes, synthetic waxes, petroleum waxes, ethylenic polymers, hydrocarbons such as Fischer-Tropsch waxes, silicone waxes, and mixtures thereof, for example. Cosmetic waxes have an emollient effect on the skin, giving a film permeable for moisture (i.e. water vapor) and gas.

Natural waxes traditionally used in cosmetic compositions include waxes of animal origin, e.g., beeswax (including beeswax derivatives such as siliconyl, cera bellina, butyl-octanyl, and hexanediol-behenyl), spermaceti, and lanolin (wool wax); waxes of vegetable origin, e.g. candelilla, carnauba, bayberry, and sugarcane wax; waxes of mineral origin, e.g. ceresin, montan, and ozokerite; and waxes of petroleum origin, e.g. paraffin and microcrystalline wax. Animal, plant, and some mineral waxes are primarily esters of a high molecular weight fatty alcohol with a high molecular weight fatty acid.

Synthetic waxes include long chained polymers of ethylene oxide combined with a dihydric alcohol, namely polyoxyethylene glycol, (including carbowax), hydrocarbon waxes derived from carbon monoxide and hydrogen (Fischer-Tropsch synthesis waxes), and silicone waxes such as methyloctadecane-oxypolysiloxane and poly(dimethylsiloxy)stearoxysiloxane.

Suitable emollients useful in a compound used to form a region of the invention include, but are not limited to, those informally classified as oils, esters, silicones, and triglycerides.

Emollients include, but are not limited to, the following compounds: 2-ethyl-hexyl succinate, butyl stearate, cetyl isooctanoate, cetyl lactate, cetyl ricinoleate, canola oil, castor oil, cocoa butter, glyceryl tri-2-ethyl-hexanate, hexadecyl alcohol, hydrogenated polyisobutene, isononyl isononanoate, isoeicosane, squalane, isopropyl palmitate, isopropyl myristate, jojoba oil, lanolin alcohol, lanolin oil, meadowfoam seed oil, mineral oil, myristyl lactate, octyldodecanol, octyldodecyl myristate, octyldodecyl oleate, octyldodecyl ricinoleate, oleic acid, oleyl alcohol, olive oil, petrolatum, phenyltrimethicone alkyl dimethicones, polybutene, propylene glycol monolaurate, stearyl isooctanoate, triglycerides, and the like. Emollients can be used either alone or in combination. Some emollients impart gloss to a composition and, thus, to the lips of a user of a lipstick including the composition. Such emollients can also be referred to as gloss components, and include compounds such as polybutene and mineral oil.

A variety of color components can be used in a composition used to form a region of a lipstick of the invention, including inorganic and organic dyes, pigments, and lakes, both oil-soluble and oil-insoluble. Generally, suitable color components include, but are not limited to, D&Cs (including Red No. 3 and Al Lake, Red No. 6 and Ba Lake, Red No. 7 and Ca Lake, Red No. 21 and Al Lake, Red No. 27 and Al Lake, Red No. 33 and Al Lake, Red No. 7, Red No. 9, Red No. 19, Red No. 21, Red No. 30, Red No. 36, Orange No. 5, Orange No. 17, Yellow No. 10), FD&Cs (including Yellow Nos. 5 and 6 and Al Lake; Blue No. 1 and Al Lake), titanium dioxide (including pigmentary and ultrafine), iron oxides (including pigmentary and ultrafine), zinc oxide (including pigmentary and ultrafine), ultramarines, magnesium violet, ferrous blue, chromium greens, and carmine. Suitable color components are available from Warner Jenkinson Inc. of St. Louis, Mo., for example.

Optionally, other agents can be added to affect the visual character of a composition forming a region of a lipstick of the invention. Such agents include pearlescent materials (including guanine, bismuth oxychloride, mica, titanium dioxide coated mica, and iron oxide coated mica), flourescent agents, and glitter and sparkle agents (which are discrete particles that can be made from coated plastics). Generally, these agents can be added to a composition forming a region of a lipstick of the invention in the same concentrations as color components.

Flavor and fragrance ingredients suitable for use in cosmetic compositions can be used in a composition that forms a region of a lipstick of the invention. At low levels of flavor and fragrance components, their presence is imperceptible to the human senses, whereas at elevated levels flavor and fragrance components may act as irritants to human skin. Thus, at least one of a suitable flavor and fragrance component preferably is included in the composition at about 0% to about 10% by weight of the composition, more preferably about 0% to about 2%, for example 1% by weight of the composition. A variety of suitable flavor ingredients are available from Medallion International of Riverdale, N.J., for example. A variety of suitable flavor and fragrance ingredients are available from Carrubba, Inc., of Milford, Conn., for example.

A particularly preferred flavor agent is sodium saccharine, preferably solvated in propylene glycol. When used in conjunction with a flavor or fragrance component, sodium saccharine functions to boost the sensory impact of the flavor or fragrance component. At lower concentrations, the effect is imperceptible, whereas at higher concentrations the effect can be overbearing. Thus, when used, sodium saccharine preferably is included at about 0% to about 0.1%, more preferably about 0.02% to about 0.06%, for example 0.05%, based on the weight of the composition. A suitable sodium saccharine is sold as a 20% solution of sodium saccharine in propylene glycol, available from Universal Preservachem Inc. of Edison, N.J.

A composition forming a region of a lipstick of the invention can include one or more preservatives. Suitable preservatives include, but are not limited to, compounds such as butylparaben, methylparaben, propylparaben, and mixtures thereof. Chelating agents, such as citric acid, and antioxidants, such as BHT, also can be used help stabilize and preserve a composition useful in the lipstick of the invention.

A composition forming a region of a lipstick of the invention can include topically active compounds including, but not limited to, topically-active drugs and medicaments, topical anesthetics, topical anti-inflammatories, topical sunscreens, UV absorbers, vitamins (including vitamins A, D, and E), antibacterial compounds, antifungal compounds, special treatment ingredients, and the like. A preferred special treatment ingredient is a peptide (palmitoyl-GHK, about 1,000 ppm) in a mixture of octyl palmitate, tribehenin and sorbitan isostearate, which is sold in the form of an opalescent balm under the trade name MAXI-LIP by Sederma of Le Perray en Yvelines, France. Typically, topically-active compounds are added in small amounts, preferably about 10% or less, most preferably about 2% or less of the total composition by weight.

A topically-active compound can be included in an amount sufficient to perform its intended function. For example, topically-active drugs, such as analgesics; antibacterials and antiseptics; antifungal compounds; anti-inflammatory compounds; antiparasitics; topical anesthetics; burn relief ointments; depigmenting agents; dermatologicals; diaper rash relief agents; skin rash, skin disease and dermatitis medications; herpes treatment drugs; pruritic medications; psoriasis, seborrhea and scabicide agents; and anti-itch and irritation-reducing compounds can be incorporated in a composition used to form a region of the lipstick of the invention in an amount sufficient to perform its intended function.

A safe and effective amount of sunscreen agent and/or a UV absorber can be used in a composition forming a region of a lipstick of the invention. Thus, when used, at least one of a sunscreen agent and a UV absorber preferably is included at about 0.1% to about 10%, more preferably from about 1% to about 5%, by weight of the composition. Exact amounts will vary depending upon the agent chosen and the desired Sun Protection Factor (SPF).

Suitable sunscreen compositions for use on human skin are known to those in the art and include, but are not limited to, octyl methoxycinnamate, benzophenone-3, butyl methoxydibenzoylmethane, oxybenzone, PABA and PABA derivatives. Various other sunscreen materials are found in “A Survey of Ultraviolet Absorbers in Commercially Available Sun Products,” by Roelandts, et al., International Journal of Dermatology, Vol. 22, pages 247-55 (May 1985) and the CTFA Cosmetic Ingredient Handbook, J. M. Nikitakis, Ed., 1st Edition, pages 86-87 (1988), the disclosures of which are hereby incorporated herein by reference. A composition forming a region of a lipstick of the invention can also include a diester and/or polyester of a naphthalene dicarboxylic acid that photostabilizes sunscreen components.

Suitable UV absorbers are known to those in the art and include, but are not limited to, allantoin PABA, butyl methoxydibenzoylmethane, ethyl diisopropylcinnamate, octyl methoxycinnamate, octyl salicylate, and PABA. Various other UV absorbers can be found in the CTFA Cosmetic Ingredient Handbook, J. M. Nikitakis, Ed., 1st Edition, page 98 (1988), the disclosure of which is hereby incorporated herein by reference. Octyl salicylate is preferred. Suitable UV absorbers components include the ESCALOL family of UV absorbers, such as ESCALOL 557 (octyl methoxycinnamate), ESCALOL 587 (octyl salicylate), and ESCALOL Z-100 (zinc oxide, octyl methoxycinnamate, PVP/hexadecene copolymer and methicone) sold by International Specialty Products of Wayne, N.J.

Preferably, at least one region of the lipstick of the invention is opaque. Most preferably, at least one region of the lipstick of the invention is opaque and includes a color component. The primary components of such an opaque composition are a structure-providing component, such as a structural wax (e.g., carnauba), a color component, such as an organic pigment (e.g., D&C Red No. 7) and, when necessary, a liquid, such as an oil (e.g., castor oil), which can also be used to wet out the pigment.

Preferred opaque compositions with color components include, but are not limited to, compositions suitable as lip rouges, lip liners, and the like, including such compositions already known and used in the art. Lip rouge compositions are most preferred.

Any composition suitable for use as a lip rouge can be used to provide a region of the lipstick of the invention, and can include any of the components identified above, such as such as antioxidants, preservatives, flavors, fragrances, glitter components, sparkle components, vitamins, sunscreens, UV absorbers, proteins, special treatment ingredients, and mixtures thereof.

One formulation of a lip rouge composition that can form a region of a lipstick of the invention is provided below. Standardized names according to the Cosmetic, Toiletry, and Fragrance Association (CTFA) or International Nomenclature of Cosmetic Ingredients (INCI) are used. Suitable suppliers of various components are provided, and supplier brand names or designations are provided when they differ from the CTFA/INCI component name. All percentages are by weight.

| CTFA/ | Supplier designation | | % of |
|---|---|---|---|
| INCI Name | or BRAND NAME | Supplier | Total |
| carnauba | Carnauba Wax T-1 | Strahl & Pitsch, Inc. | 1.66 |
| | | West Babylon, NY | |
| castor oil | Triglyceride of | Hansotech, Inc. | 28.79* |
| | Ricinoleic Acid | Woodbury, NY | |
| candelilla | Candelilla Wax | Strahl & Pitsch, Inc. | 8.65 |
| | S.P. 75 | West Babylon, NY | |
| polybutene | INDOPOL H-100 | Amoco Chemical Co. | 5.00 |
| | | Chicago, IL | |
| propylene | SCHERCEMOL | Scher Chemicals, Inc. | 7.56 |
| glycol laurate | PGML | Clifton, NJ | |
| octyl dodecanol | EUTANOL G | Cognis Corp. | 6.22 |
| | | Ambler, PA | |
| hydrogenated | Dub PP H-1/ | M.M.P., Inc. | 6.50 |
| palm kernel | PALMERIDE | Plainfield, NJ | |
| glycerides | | | |
| isononyl | DERMOL 99 | Alzo, Inc. | 2.18 |
| isononanoate | | Matawan, NJ | |
| methylparaben | | Acme-Hardesty | 0.20 |
| | | Blue Bell, PA | |
| propylparaben | | Acme-Hardesty | 0.10 |
| | | Blue Bell, PA | |
| butylparaben | | Acme-Hardesty | 0.05 |
| | | Blue Bell, PA | |
| citric acid | | Alfa Chem | 0.03 |
| | | Kings Point, NY | |
| BHT | butylated | Eastman | 0.06 |
| | hydroxytoluene | Kingsport, TN | |
| color | | | 30.00 |
| components | | | |
| flavors | | | 1.00 |

*Q.S. castor oil due to topically-active compounds.

In the formulation given above, the amount of castor oil preferably is reduced in proportion to the amount of topically-active compounds added. Topically-active compounds include, but are not limited to, those previously mentioned, such as vitamins, sunscreens, proteins, and special treatment ingredients.

The formulation described in the table above includes lower concentrations of emollients, particularly oils, than typical lip rouge compositions and is particularly suited for forming a first region of a lipstick of the invention when a second region of the lipstick of the invention is made up of a highly emollient composition, such as a preferred polyamide resin-based translucent or transparent composition described below, which has a high concentration of emollients, particularly oils. In this manner, the two compositions can blend upon application to the lips and lay down a blended layer of lip cosmetic having a desirable level of emolliency. Preferably, the amount of emollients in a first region of a lipstick is any amount between 0% and less than about 55% by weight, more preferably between 0% and about 50%, for example about 48% by weight of the composition forming the region.

Furthermore, a highly emollient composition used to form a region of the lipstick preferably has a wax concentration lower than in typical lip cosmetic compositions. In an highly emollient composition used to form a region of the lipstick, waxes preferably are included in any amount between 0% and about 20% by weight, more preferably between 0% and less than about 10% by weight, and most preferably the composition is essentially free of waxes.

A composition useful for forming a region of the lipstick of the invention is described in commonly-owned U.S. patent application Ser. No. 09/597,474, filed Jun. 20, 2000, the disclosure of which is incorporated herein by reference. An embodiment of that invention preferred for use as a translucent or transparent region of the lipstick of this invention will now be described in detail.

A composition useful for forming a translucent or transparent region in a lipstick of the invention includes a polyamide resin, a solvent for the polyamide resin, and a gelling agent selected from the group consisting of an N-acyl amino acid amide, an N-acyl amino acid ester, and mixtures thereof. Preferably, the composition is essentially free of volatile alcohols, which eliminates the need for specialized packaging and containers, eliminates hardening of the composition caused by evaporation of volatile alcohols, and eliminates the need for added flavor and fragrance components otherwise used to mask the taste and smell of alcohol.

The polyamide resin provides the composition with structure, while allowing the composition to have a translucent or transparent appearance.

As the amount of resin is decreased, the composition begins to lose clarity, while as the amount of resin is increased, the composition takes on an undesirable taste, which can be detrimental to a lipstick product. As the amount of resin is increased, the composition also becomes hard and loses the ability to rub off onto the lips or skin of a user. Thus, for a region in a lipstick of the invention, the polyamide resin preferably is present in the composition at about 5% to about 15%, more preferably about 7% to about 12%, based on the total weight of the region.

The polyamide resin must be soluble in a cosmetically-acceptable solvent at elevated temperatures and must solidify (e.g., form a gel solid) upon cooling. The polyamide resins that are useful in the invention preferably should be soluble in a suitable cosmetically-acceptable solvent at a temperature between about 50° C. and about 150° C., most preferably between about 50° C. and about 130° C. Given this temperature range, typically the preferred polyamide resins are not extensively covalently crosslinked, which would limit or prevent solubility. Suitable polyamide resins for use in the invention are classified as thermoplastics rather than thermosets.

Preferred polyamide resins are based on complex fatty acids, for example the VERSAMID series sold by Henkel Corp. of Ambler, Pa. or the UNIREZ series sold by Union Camp Corp. Polyamide resins based on terpolymers of simple nylons, such as Dupont Corporation's ELVAMIDE 8061, which is a terpolymer of nylon 6, nylon 66, and nylon 610, may also be suitable for use in the invention, for example when they are soluble in suitable cosmetically-acceptable solvents at a temperature between about 50° C. to about 150° C.

A preferred polyamide resin based on complex fatty acids includes a condensation product of a polycarboxylic acid with a diamine (e.g., ethylenediamine, propylenediamine, or hexylenediamine), most preferably aliphatic diamines. Preferably, the polycarboxylic acid is a dicarboxylic acid (e.g., adipic acid, oxalic acid, sebacic acid, and maleic acid). Polyamide resins are described in Kirk-Othmer Encyclopedia of Chemical Technology, 4th Ed. (1993) at volume 8, pages 232-233, and in Ross et al. U.S. Pat. No. 5,500,209 (Mar. 19, 1996), the disclosures of which are hereby incorporated herein by reference.

The polyamide resin is a solid at room temperature, preferably composed primarily of polyamides of weight average molecular weight or weights in the range of about 1,000 to about 30,000 daltons, most preferably about 2,000 to about 10,000 daltons.

Preferred resins are the VERSAMID resins, particularly VERSAMID 930 polyamide resin, which is a condensation product of adipic acid and hexylenediamine. When using a polyamide resin such as VERSAMID, the polyamide resin preferably is present at about 5% to about 12% by weight of the composition, most preferably about 6% to about 10%, for example 8% by weight of the composition.

A suitable solvent for the polyamide resin modifies the polyamide resin to soften the resin and provide a composition with the desired firmness and transfer (ruboff) characteristics. A solvent for the polyamide resin can also function as a gloss agent, an emollient, a viscosity modifier, a vehicle for an optional component such as color, flavor, fragrance, sunscreen, and vitamin, and a co-solvent for components of the composition. The solvent for the polyamide need not be a single solvent, but also can be a solvent system including a plurality of solvents.

A suitable type and amount of solvent for the polyamide resin preferably are selected such that the polyamide resin can be dissolved therein at elevated temperatures, and yet can form a solid upon cooling. Preferably, the type and amount of solvent for the polyamide resin are selected such that a film of the polyamide resin-based composition can be transferred from the surface of the composition to the lips or skin of the user without compromising the structural integrity of the composition.

As the amount of the solvent is decreased, the appearance of the composition can change from transparent to translucent to opaque. The tactile characteristics of the composition also can change as the amount of solvent is decreased, such that the composition loses emolliency, becomes hard, and loses ruboff ability. When decreasing the content of certain solvents (e.g., polybutene and octyldodecanol), the composition also can lose a desirable wet, glossy appearance.

As the amount of solvent is increased, a detrimental result common with most solvents is the loss of structural integrity of the composition. As the amount of solvent is increased, the composition may also exhibit undesirable tactile characteristics, such as tackiness in the case of a polybutene solvent. As the amount of certain solvents is increased, the appearance of the composition can change from transparent to translucent (such as in the case of octyldodecanol), and even opaque.

Thus, a solvent for the polyamide preferably is included in the composition at about 46% to about 97%, more preferably about 65% to about 97%, based on the total weight of the composition.

The solvent for the polyamide preferably is selected from the group consisting of unsaturated fatty alcohols (10-20 carbon atoms, branched or straight chain), such as oleyl alcohol or ricinoleic alcohol; saturated fatty alcohols (8-20 carbon atoms, branched or straight-chain), such as myristyl alcohol, lauryl alcohol, isolauryl alcohol, isostearyl alcohol, and isocetyl alcohol; fatty and/or aromatic carboxylic acid esters, such as benzyl benzoate, isostearyl benzoate, C₁₂-C₁₅ alkyl benzoates, C₁₀-C₁₅ alkyl lactates (including lauryl lactate), propylene glycol monolaurate, polyethylene glycol (400) monolaurate, liquid castor oil, isopropyl myristate, isopropyl palmitate, propyl myristate with the general formula RCOOR′, where R and R′ can be the same or different, and are from 2 to 20 carbon atoms, and can be saturated, unsaturated or aromatic; ethoxylated and/or propoxylated alcohols and acids, such as PPG-14 myristyl ether, PPG-14 butyl ether, PPG-3 myristyl ether, and myristeth-3 propionate; silicones such as cyclomethicones, dimethicones (50 to 1,000,000 cps) and functional silicones; mineral oils; branched-chain hydrocarbons, such as those sold under the trade name ISOPAR from Exxon Corporation, and those sold as PERMETHYL by Presperse, Inc., of South Plainfield, N.J. (e.g., isoeicosane, sold as PERMETHYL 102A); and mixtures thereof. Solvents that impart gloss to a composition and, thus, to the lips of a user of a lipstick including the composition are particularly preferred.

Castor oil [CAS registry # 8001-79-4] (liquid castor oil) is described in Kirk-Othmer Encyclopedia of Chemical Technology, 4th Ed. (1993) at volume 5, pages 301-320, the disclosure of which is hereby incorporated herein by reference. Castor oil is a triglyceride of various fatty acids, substantially all of which are unsaturated fatty acids. Castor oil esters are changed by hydrogenation from liquid products to soft waxes having melting points of about 45° C. to about 80° C. A castor oil suitable for use in a preferred translucent or transparent composition to form a region of the lipstick of the invention is a liquid product at room temperature, most preferably non-hydrogenated. Such a castor oil is sold under its common name by Hansotech, Inc. of Woodbury, N.Y.

A solvent preferably is selected from the group consisting of fatty acid esters, fatty alcohols, mineral oils, branched-chain hydrocarbons, and mixtures thereof.

More preferably, a solvent for the polyamide resin is selected from the group consisting of liquid castor oil, propylene glycol monolaurate, polyethylene glycol (400) monolaurate, lauryl lactate, oleyl alcohol, isoeicosane, polybutene (also known as polyisobutylene, polybutylene, and polyisobutene) octyldodecanol, and mixtures thereof. A polybutene product suitable for use in the invention is sold under the name INDOPOL H-100 by Amoco Chemical Company of Chicago, Ill., and is an isobutylene/butene copolymer (CAS # 9003-29-6). An octyldodecanol (CAS # 5333-42-6) product suitable for use in the invention is sold under the name EUTANOL G by Cognis Corporation of Ambler, Pa., and contains 2-octyl dodecanol as a principal component. A fatty ester product suitable for use in the invention is sold under the name ULTRACAS G20 by B. F. Goodrich Performance Materials of Cleveland, Ohio. A particularly preferred blend of solvents includes liquid castor oil, polybutene, isoeicosane, and, optionally, octyldodecanol.

When a blend of solvents including liquid castor oil, polybutene, and isoeicosane is used as the solvent for the polyamide resin, liquid castor oil preferably is added at about 10% to about 40%, more preferably about 13% to about 28%; isoeicosane preferably is added at about 35% to about 45%, more preferably about 37% to about 43%; and polybutene preferably is added at about 1% to about 10%, more preferably about 3% to about 7%; all based on the weight of the composition. Optionally, the solvent for the polyamide resin includes octyldodecanol, preferably at about 15% to about 25%, more preferably at about 18% to about 23%, based on the weight of the composition.

It is theorized, but not relied upon herein, that the gelling agent acts to gel one or more components including an oil or solvent that are released from the polyamide resin as the temperature of the composition increases, and that the gelling agent subsequently releases gelled components for reabsorption back into the polyamide resin matrix upon cooling. Thus, it is theorized that as the amount of solvent in the composition increases, the amount of gelling agent should be increased in order to obtain the full advantages of the invention. Similarly, it is theorized that as the amount of solvent in the composition decreases, the amount of gelling agent could be decreased to avoid certain disadvantages such as reduction in transparency, and for cost savings.

As the amount of gelling agent in the composition decreases, composition loses the ability to resist irreversible syneresis. Thus, it is theorized that when a composition containing a polyamide resin and solvent and low concentrations of gelling agent is subjected to an increased temperature environment, components of the composition including oils irreversibly migrate out of the resin matrix.

On the other hand, as the amount of gelling agent in the composition increases, the composition can change from transparent to translucent and even opaque. Moreover, using more than a suitable amount of gelling agent provides no additional benefit from a syneresis standpoint, and only serves to increase cost of the composition.

Thus, a gelling agent preferably is included in the composition at about 0.1% to about 10%, more preferably about 3% to about 7%, based on the total weight of the composition used to form a region of the lipstick of the invention.

The gelling agent is selected from the group consisting of N-acyl amino acid amides, N-acyl amino acid esters, and mixtures thereof. For example, N-acyl glutamic acid diamide is sold as oil gelatinization agent GP-1 by Anjinomoto Co. Inc., of Tokyo, Japan. Generally, N-acyl amino acid gelling agents are described in Saito et al. U.S. Pat. No. 3,969,087 (Jul. 13, 1976), the disclosure of which is hereby incorporated herein by reference.

Preferably, the gelling agent has the formula:

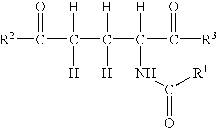

wherein R¹ is an alkyl, aryl, aralkyl radical having from about 6 to about 22 carbon atoms, and R² and R³, independently, are an alkyl, aryl, aralkyl ester radical or amide radical in which the alkyl, aryl, aralkyl moiety has from about 2 to about 20 carbon atoms.

The term “alkyl” as used herein is a hydrocarbon group containing the indicated number of carbon atoms and includes straight chained and branched alkyl or alkylene groups, typically methyl, ethyl, and straight chain and branched propyl, propylene, and butyl groups.

The term “aryl” as used herein refers to optionally substituted 5- or 6-membered carbocyclic and heterocyclic aromatic groups, including, but not limited to, phenyl, thienyl, furyl, pyrryl, imidazolyl, pyrimidyl, and pyridyl. The term “aralkyl” as used herein refers to an aryl group having a C₁₋₁₅ alkyl substituent.

More preferably, a gelling agent is selected from the group consisting of N-lauroyl glutamic acid diethylamide, N-lauroyl glutamic acid dibutylamide, N-lauroyl glutamic acid dihexylamide, N-lauroyl glutamic acid dioctylamide, N-lauroyl glutamic acid didecylamide, N-lauroyl glutamic acid ditetradecylamide, N-lauroyl glutamic acid dihexadecylamide, N-lauroyl glutamic acid distearylamide, N-stearoyl glutamic acid dibutylamide, N-stearoyl glutamic acid dihexylamide, N-stearoyl glutamic acid diheptylamide, N-stearoyl glutamic acid dioctylamide, N-stearoyl glutamic acid didecylamide, N-stearoyl glutamic acid didodecylamide, N-stearoyl glutamic acid ditetradecylamide, N-stearoyl glutamic acid dihexadecylamide, N-stearoyl glutamic acid distearylamide, and mixtures thereof.

Most preferably, the gelling agent includes N-lauroyl glutamic acid dibutylamide.

One preferred temperature-stable translucent or transparent composition includes about 3 wt. % to about 15 wt. % of a polyamide resin; about 10 wt. % to about 40 wt. % of liquid castor oil; about 35 wt. % to about 45 wt. % of isoeicosane; about 1 wt. % to about 10 wt. % of polybutene; and about 0.1 wt. % to about 20 wt. % of a gelling agent having the formula:

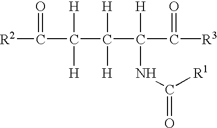

wherein R¹ is an alkyl, aryl, aralkyl radical having from about 6 to about 22 carbon atoms, and R² and R³, independently, are an alkyl, aryl, aralkyl ester radical or amide radical in which the alkyl, aryl, aralkyl moiety has from about 2 to about 20 carbon atoms.

In addition to the polyamide resin, solvent, and gelling agent, a preferred composition useful as a translucent or transparent region in a lipstick of the invention optionally and preferably includes a surfactant (surface active agent), which can also be a blend of surfactants. The surfactant acts as a viscosity modifier or thickener, reduces the susceptibility of the composition to syneresis, and improves the texture of the composition.

Thus a surfactant preferably is included in the composition at about 0% to about 20%, and more preferably about 1% to about 12%, by weight of the composition. Preferably the surfactant is a nonionic surfactant or a nonionic surfactant blend having an HLB value of about 3 to about 20. Most preferably, the surfactant is present in an amount of about 3% to about 8%, by weight of the composition.

A preferred composition useful as a translucent or transparent region of the invention also can comprise additional, optional components to provide desirable properties. Suitable optional ingredients include, but are not limited to, preservatives, color, flavor, and fragrance components, pearlescent agents, glitter agents, sparkle agents, sunscreens, UV absorbers, vitamins, texture enhancers, fillers, and other suitable agents.

In certain cases, it may be desirable to add lower aliphatic alcohols to the composition to further inhibit syneresis, for example in products that do not permit or require reuse after breaking the seal of a package or container, or in special use environments or products where syneresis may be an acute problem. In such cases, a lower aliphatic alcohol (C₁-C₈, branched or straight-chain) preferably is used in the composition of the invention at 0% to about 10%, more preferably about 0.1% to about 5%, most preferably less than 2%, by weight of the composition. A lower aliphatic alcohol suitable for use in a composition of the invention is isopropyl alcohol, sold under its common name as a 99% solution by Research Solvents and Chemicals, Inc.

When a preservative is used in a preferred composition used to form a translucent or transparent region in a lipstick of the invention, it is preferably is included in the composition at about 0.01% to about 0.5%, more preferably about 0.05% to about 0.2%, for example 0.1%, by weight of the composition. As the concentration of preservative is increased, it is increasingly difficult to solubilize the preservative and the preservative may irritate the skin, whereas when the concentration of preservative is decreased it becomes less effective.

The above-described composition used to form a translucent or transparent region of a lipstick of the invention typically has a slight yellow tinge in the absence of color agents, and can be formulated to be highly transparent. At elevated amounts of color components, the composition can change from transparent to translucent and even opaque. Thus, to make a translucent or transparent composition, a suitable color agent preferably is included in the composition at about 0% to about 2%, more preferably about 0% to about 0.5%, for example 0.02%, by weight of the composition.

Preferred color components include FD&C Yellow No. 5 Al Lake, FD&C Yellow No. 6 Al Lake, D&C Red No. 28 Al Lake, D&C Red No. 6, and FD&C Blue No. 1 Al Lake.

When one or more of a flavor and fragrance component is added to the composition, it is preferably included at about 0% to about 5%, more preferably about 0% to about 2%, for example 1%, by weight of the composition. At low levels of flavor and fragrance components, their presence is imperceptible to the human senses, whereas at elevated levels, the composition could be detrimentally affected by a change from transparent to translucent and even opaque and by loss of structural integrity.

It may be desirable to include one or more vitamins to the preferred translucent or transparent composition. At low concentrations, vitamins generally are ineffective, whereas at higher concentrations a composition of the invention can be detrimentally affected by a change from transparent to translucent and even opaque and by loss of structural integrity. Thus, when used, vitamin preferably is included at about 0.01% to about 5%, more preferably about 0.1% to about 3%, for example 1%, based on the weight of the composition. Preferred vitamins include oil-soluble vitamins, such as vitamin A, vitamin D, and vitamin E.

When one or more of a sunscreen and a UV absorber is used in the preferred translucent or transparent composition, it preferably is included at about 0.1% to about 10%, more preferably from about 1% to about 5%, by weight of the composition. At low concentrations, sunscreen agents and UV absorbers are less effective, whereas at higher concentrations a composition of the invention can be detrimentally affected by a change from transparent to translucent and even opaque and by loss of structural integrity.

A preferred formulation of a translucent polyamide resin-containing composition that can form a region of a lipstick of the invention is provided below. Standardized names according to the Cosmetic, Toiletry, and Fragrance Association (CTFA) or International Nomenclature of Cosmetic Ingredients (INCI) are used. Suitable suppliers of various components are provided, and supplier brand names or designations are provided when they differ from the CTFA/INCI component name. All percentages are by weight.

| CTFA/ | Supplier designation | | % of |
|---|---|---|---|
| INCI Name | or BRAND NAME | Supplier | Total |
| castor oil | Triglyceride of | Hansotech, Inc. | 18.3 |
| | Ricinoleic Acid | Woodbury, NY | |
| octyl dodecanol | EUTANOL G | Cognis Corp. | 21.17 |
| | | Ambler, PA | |
| polybutene | INDOPOL H-100 | Amoco Chemical Co. | 5.00 |
| | | Chicago, IL | |
| | VERSAMID 930 | Henkel Corp. | 8.00 |
| | | Ambler, PA | |
| | GP-1 | Ajinomoto Co. Inc. | 5.00 |
| | | Tokyo, Japan | |
| sorbitan oleate | LIPOSORB O | Lipo Chemicals, Inc. | 2.50 |
| | | Paterson, NJ | |
| polysorbate 80 | LIPOSORB O-20 | Lipo Chemicals, Inc. | 2.50 |
| | | Paterson, NJ | |
| propylparaben | | | 0.1 |
| isoeicosane | PERMETHYL 102 | Presperse Inc. | 36.35* |
| | | South Plainfield, NJ | |
| color | | | about |
| components | | | 0.01-0.10 |
| flavors | | | 1.00 |

Q.S. isoeicosane due to color component concentration

In the formulation given above, the amount of isoeicosane preferably is reduced in proportion to the amount of color components added.

It has been discovered that certain translucent or transparent polyamide resin-based compositions according to the description above can take on a cloudy, near-opaque appearance after prolonged contact with a certain white plastic lipstick case. Although the mechanism is unknown, the phenomenon appears to be caused by the presence of polyoxymethylene (also known as polyacetal or acetal copolymer) and, thus, the use of this compound should be avoided in cases used with lipsticks of the invention containing a polyamide resin-based composition according to the description above when translucency or transparency is desired. The phenomenon does not appear to occur when using general purpose polystyrene lipstick cases.

Another aspect of the invention is achieved when a first region of the lipstick of the invention includes a first flavor component and a second region of the lipstick includes a second flavor component. By keeping different flavors substantially isolated, a unique taste experience is achieved, whereas if different flavors are mixed and incorporated into a lipstick composition, the flavor blend can be confusing to the user's taste buds. In one embodiment of this aspect of the invention, a first region of a lipstick (e.g., formed from an opaque lip rouge composition) includes an orange flavor component and a second region (e.g., a transparent composition) includes a vanilla flavor component. Application of the resulting lipstick provides the user with a creamsicle-type taste sensation not achievable by a simple blending of orange and vanilla flavor components into one composition. Other flavor combinations (including combinations with more than two flavors) will be apparent to those of skill in the art, such as cherry/chocolate, mint/chocolate, raspberry/vanilla, and the like.

This aspect of the invention is further enhanced when the flavor component in a first region resists migration into a second. region of the lipstick. When a preferred polyamide resin-based composition as described herein for use as a translucent or transparent region is used in the lipstick of the invention, isolation (i.e., resistance to migration) of flavor components is enhanced.

Structural stability of a lipstick of the invention is enhanced when the melting points of the various regions of the lipstick are close to one another. Thus, another aspect of the invention is a lipstick wherein a first region has a melting point less than about 15° C. different from a the melting point of a second region, preferably less than about 12° C. different, most preferably less than about 10° C. Preferably, each region of a lipstick of the invention has a respective melting point less than about 12° C. different from all other regions. Too great a difference in melting points can cause one region to expand or contract with respect to another region when the lipstick is exposed to elevated temperatures.

Structural stability of a lipstick of the invention is further enhanced when a higher-melting composition provides an outer surface of the lipstick. For example, in a lipstick of the core-sheath type, it is preferred to formulate the sheath material with a higher melting point than the core material. When a preferred polyamide resin-based composition as described herein for use as a translucent or transparent region is used in the lipstick of the invention, preferably the concentration of polybutene is raised or lowered to control (decrease or increase, respectively) the melting point of the composition.

A lipstick according to the invention can be provided with any shape desired by any suitable process. Preferably, a lipstick of the invention is shaped by molding, and is provided with a classic bullet shape and beveled upper portion.

The various regions of the lipstick of the invention can be disposed in any configuration to provide the appearance desired. Preferably, the regions of the lipstick are contiguous, but the invention is not so limited. Most preferably the regions of a lipstick of the invention are substantially coextensive lengthwise of the lipstick. Suitable non-limiting examples of lipsticks having a plurality of regions can be found in the prior art, such as core-sheath type lipsticks described in U.S. Pat. No. 4,291,018 and Japanese Patent Publication No. 61-112008, side-by-side arrangements described in U.S. Pat. No. 4,743,443 and Japanese Patent Publication No. 61-112008, and marbleized and swirled arrangements described in U.S. Pat. No. 3,479,429 (Nov. 18, 1969), each of which is incorporated herein by reference. Various other configurations can be used, limited only by the imagination of the fabricator.

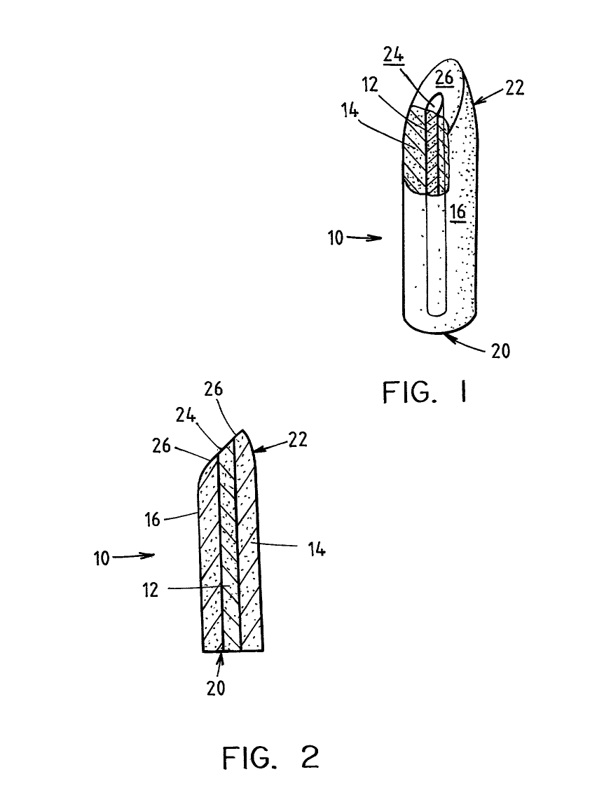

A preferred configuration of an article of the invention will now be described with reference to the Figures. FIG. 1 is a partially broken-away perspective view of an embodiment of the invention wherein the lipstick is the core-sheath type. FIG. 2 is a cross-sectional elevational view of an embodiment of the invention wherein the lipstick is the core-sheath type. Like reference numbers in the figures refer to like elements. With reference to FIG. 1, the lipstick, generally designated 10, includes two regions disposed in core-sheath relationship along a longitudinal axis of the lipstick 10. A core region 12 is surrounded by a sheath region 14, the sheath region 14 defining an outer surface 16 of the lipstick 10. FIG. 2 shows that the embodiment has a flat (planar) bottom portion 20 and a beveled upper portion 22. FIGS. 1 and 2 show that the core region 12 is exposed at the face of the lipstick 10 at the upper portion 22 of the lipstick 10 (i.e., the upper axial end), the exposed face indicated by reference number 24. The exposed face of the sheath region 12 bears reference number 26. In an embodiment of the invention wherein the lipstick is of the core-sheath type such as the lipstick 10 in FIGS. 1 and 2, preferably the sheath region 12 is translucent or transparent, and the core region 14 is visible through the sheath region 12, providing a unique, high-fashion appearance.

Examples of other configurations include lipsticks wherein regions are disposed side-by-side in radial fashion, such as a candy-stripe configuration or barber pole-type configuration. Providing at least one transparent region allows the fabricator to produce unique, high-fashion, and highly functional lipsticks of the invention. Core-sheath type lipsticks can also be provided with variations, wherein the cross-sectional shapes of the core and sheath can be, same or different, selected from shapes such as circular, elliptical, oval, triangular, square, pentagonal, hexagonal, and rhombic. A substantially concentric construction is preferred for structural stability reasons.

When a first region includes a color component and a second region contains a gloss component, preferably the regions are disposed in a configuration that permits blending of the compositions forming the regions upon application to the lips in a single application step. Preferably, the top end of the lipstick is formed with a smooth surface (face) intersecting the longitudinal axis of the lipstick, most preferably inclined to the longitudinal axis thereof, such as in the case of a beveled end.

A preferred core-sheath type lipstick of the invention includes a lip rouge composition as a core region and a polyamide resin-based composition as a translucent or transparent sheath region. Such a lipstick can be formed by any suitable method, including molding. Preferably, the lipstick is formed by sequential molding of each region in a process wherein a portion of the mold cavity is occupied by a removable insert (i.e., so that a portion of the mold cavity is prevented from filling) in a first filling operation, the insert is then removed, and then second filling operation fills in the void previously occupied by the removable insert. Use of a volatile silicone spray release agent (e.g. Dow D4 silicone spray) can assist in demolding.

In one embodiment of the invention wherein the lipstick is of the core-sheath type, the preferred mold has a cylindrical cavity with a diameter of about 0.477 inches and a length of about 1.50 inches. In a preferred embodiment wherein the sheath is molded first, the removable insert comprises a tapered cylindrical pin having an average diameter of about 0.25 inches, known as a #4 pin in the art. Preferably, the pin is mounted on a removable base plate which, when brought together with the mold, seals the mold at one end for filling. Preferably, the mold includes a series of cavities and the matching base place includes a series of pins to facilitate production of multiple units in a single set of molding operations. The bottom of the mold can be provided with various different shapes that provide the molded lipstick with upper portions with various shapes, e.g., beveled, fish fin, and the like. After removal of the pin, a base plate having a shape corresponding to the desired upper portion of the lipstick is inserted prior to filling the composition for the core region.

### EXAMPLE

The following example is provided to illustrate the invention but is not intended to limit the scope of the invention.

A lipstick of the core-sheath type was made from a lip rouge composition and a translucent polyamide resin-containing composition.

The formulation of the rouge composition used as the core region of the lipstick is provided below. Supplier brand names or designations are provided when they differ from the CTFA/INCI component name. All percentages are by weight.

| CTFA/ | Supplier designation | | % of |
|---|---|---|---|
| INCI Name | or BRAND NAME | Supplier | Total |
| carnauba | Carnauba Wax T-1 | Strahl & Pitsch, Inc. | 1.66 |
| | | West Babylon, NY | |
| castor oil | Triglyceride of | Hansotech, Inc. | 34.79 |
| | Ricinoleic Acid | Woodbury, NY | |
| candelilla | Candelilla Wax | Strahl & Pitsch, Inc. | 8.65 |
| | S.P. 75 | West Babylon, NY | |
| polybutene | INDOPOL H-100 | Amoco Chemical Co. | 5.00 |
| | | Chicago, IL | |
| propylene | SCHERCEMOL | Scher Chemicals, Inc. | 7.56 |
| glycol laurate | PGML | Clifton, NJ | |
| octyl dodecanol | EUTANOL G | Cognis Corp. | 6.22 |
| | | Ambler, PA | |
| hydrogenated | Dub PP H-1/ | M.M.P., Inc. | 6.50 |
| palm kernel | PALMERIDE | Plainfield, NJ | |
| glycerides | | | |
| isononyl | DERMOL 99 | Alzo, Inc. | 2.18 |
| isononanoate | | Matawan, NJ | |
| methylparaben | | Acme-Hardesty | 0.20 |
| | | Blue Bell, PA | |
| propylparaben | | Acme-Hardesty | 0.10 |
| | | Blue Bell, PA | |
| butylparaben | | Acme-Hardesty | 0.05 |
| | | Blue Bell, PA | |
| citric acid | | | 0.03 |
| BHT | | Eastman | 0.06 |
| | | Kingsport, TN | |
| titanium | | Whittaker, Clark and | 2.00 |
| dioxide | | Daniels | |
| | | South Plainfield, NJ | |
| | RONA Superpearl | EM Industries, Inc. | 10.0 |
| | | Hawthorne, NY | |
| | TIMICA Gold | Englehard Corp. | 12.0 |
| | Sparkles | Iselin, NJ | |
| | vanilla flavor | Medallion International | 1.00 |
| | | Riverdale, NJ | |

The lip rouge core composition was compounded by charging all waxes and oils into a kettle and melting until clear. Next, the preservatives and color components were added with mixing, and mixed until homogeneous. The resulting mixture was passed through a roller mill three times and then stored until used for molding.

The formulation of the translucent polyamide resin-containing composition used as the sheath region of the lipstick is provided below. Supplier brand names or designations are provided when they differ from the CTFA/INCI component name. All percentages are by weight.

| CTFA/ | Supplier designation | | % of |
|---|---|---|---|
| INCI Name | or BRAND NAME | Supplier | Total |
| castor oil | Triglyceride of | Hansotech, Inc. | 18.3 |
| | Ricinoleic Acid | Woodbury, NY | |
| octyl dodecanol | EUTANOL G | Cognis Corp. | 21.17 |
| | | Ambler, PA | |
| polybutene | INDOPOL H-100 | Amoco Chemical Co. | 5.00 |
| | | Chicago, IL | |
| | VERSAMID 930 | Henkel Corp. | 8.00 |
| | | Ambler, PA | |
| | GP-1 | Ajinomoto Co. Inc. | 5.00 |
| | | Tokyo, Japan | |
| sorbitan oleate | LIPOSORB O | Lipo Chemicals, Inc. | 2.50 |
| | | Paterson, NJ | |
| polysorbate 80 | LIPOSORB O-20 | Lipo Chemicals, Inc. | 2.50 |
| | | Paterson, NJ | |
| propylparaben | | Acme-Hardesty | 0.1 |
| | | Blue Bell, PA | |
| isoeicosane | PERMETHYL 102 | Presperse Inc. | 35.93 |
| | | South Plainfield, NJ | |
| FD&C Yellow | | Warner Jenkinson | 0.50 |
| No. 6 | | St. Louis, MO | |
| | orange flavor | Medallion International | 1.00 |
| | #4-159 | Riverdale, NJ | |

The translucent polyamide resin-containing composition was compounded by charging the castor oil, octyldodecanol, and polybutene into a kettle set to 130° C. The contents of the kettle were mixed with heat until homogeneous. During mixing, when the temperature rose above 90° C., the VERSAMID 930 polyamide resin was added. When the solution became clear (with a slight yellow tint), the GP-1 gelatinization agent was added with continued mixing. When the solution became clear, the sides of the kettle and mixing blade were scraped to ensure that all components were in solution and dissolved and the solution became clear again. Next, the temperature setting was reduced to 100° C. with continued mixing. When the temperature reached about 100° C., sorbitan oleate and polysorbate 80 were added and mixed until homogenous. With continued mixing, isoeicosane then was added in aliquots to ensure that the temperature of the solution did not drop below 90° C., which prevented solidification of the solution. Next, color components and propylparaben were added and mixed until homogeneous. After the color components were completely mixed (about 3 hours) and about 30 minutes before molding began, the flavor component was added. The resulting “make and fill” composition was never cooled for storage.

To mold the lipstick, a room temperature mold having multiple cavities with diameters of about 0.447 inches and #4 taper pin inserts was used. The mold was pretreated with a volatile silicone spray release agent, then filled with sheath compound having a temperature of about 90 to 95° C. The filled mold was chilled until the sheath material set, and excess mass was then scraped from the top of the mold and discarded. Next, the base plate with core pins was removed and replaced with a plain slant base plate, to give the lipstick a beveled upper portion. With the base containing sheath material at room temperature, core material at 90° C. was poured in to the mold, then chilled until set. Excess mass was scraped from the top of the mold and discarded. Finally, the lipsticks were demolded and inserted into cases.

The resulting lipsticks had a unique, high-fashion appearance wherein a white core region was visible through a translucent orange sheath. Application of the core-sheath lipstick on the lips provided a synergistic effect wherein a first portion of glossy sheath material and a portion of core material were blended during application on the lips and a second portion of glossy sheath material was applied on top. Application of the core-sheath lipstick on the lips also provided a unique creamsicle-like taste sensation resulting from a combination of vanilla flavor from the core and orange flavor from the sheath.

The foregoing description is given for clearness of understanding only, and no unnecessary limitations should be understood therefrom, as modifications within the scope of the invention may be apparent to those having ordinary skill in the art.

What is claimed is:

1. A lipstick comprising a plurality of regions wherein at least one of said regions is translucent or transparent.

2. The lipstick of claim 1 wherein said regions are contiguous.

3. The lipstick of claim 1 wherein said regions are substantially coextensive lengthwise of said lipstick.

4. The lipstick of claim 1 wherein at least one of said regions is opaque.

5. The lipstick of claim 1 wherein at least one of said regions comprises 0% to less than about 55% emollients by weight.

6. The lipstick of claim 5 wherein at least one of said regions comprises 0% to about 50% emollients by weight.

7. The lipstick of claim 1 wherein said translucent or transparent region comprises an emollient.

8. The lipstick of claim 7 wherein said emollient comprises liquid castor oil.

9. The lipstick of claim 1 wherein at least one of said regions comprises a flavor component.

10. The lipstick of claim 9 wherein a first region of said lipstick comprises a flavor component and a second region of said lipstick comprises a flavor component.

11. The lipstick of claim 10 wherein said flavor component of said first region is different from said flavor component of said second region.

12. The lipstick of claim 1 wherein at least one region comprises 0% to less than about 10% by weight of a wax.

13. The lipstick of claim 1 wherein at least one region is essentially free of waxes.

14. The lipstick of claim 13 wherein said region essentially free of waxes is translucent or transparent.

15. The lipstick of claim 1 wherein at least one of said regions comprises a color component.

16. The lipstick of claim 15 wherein at least one of said regions comprises a gloss component.

17. The lipstick of claim 16 wherein a first region comprises a color component and a second region comprises a gloss component.

18. The lipstick of claim 17 wherein said first and second regions are disposed to permit blending of said first and second regions in a single application step.

19. The lipstick of claim 18 wherein said first and second regions are adjacent and substantially coextensive lengthwise of said lipstick.

20. The lipstick of claim 18 wherein said first and second regions are disposed in core-sheath relationship along a longitudinal axis of said lipstick.

21. The lipstick of claim 1 wherein said translucent or transparent region comprises a polyamide resin.

22. The lipstick of claim 21 wherein said polyamide resin comprises a condensation product of a dicarboxylic acid and a diamine.

23. The lipstick of claim 22 wherein at least one of said regions comprises a flavor component.

24. The lipstick of claim 23 wherein said translucent or transparent region of said lipstick comprises a flavor component and a second region of said lipstick comprises a flavor component.

25. The lipstick of claim 24 wherein said flavor component of said translucent or transparent region is different from said flavor component of said second region.

26. The lipstick of claim 1 wherein a first region and a second region have respective melting points less than about 12° C. different from each other.

27. The lipstick of claim 26 wherein each of said regions has a respective melting point less than about 12° C. different from all other regions.

28. The lipstick of claim 1 wherein said translucent or transparent region defines an outer surface of said lipstick.

29. The lipstick of claim 1 wherein a first region extends lengthwise of said lipstick and is surrounded by a second region extending lengthwise of said lipstick.

30. The lipstick of claim 29 wherein said first region has a melting point and said second region has a melting point wherein the melting point of said first region is less than or equal to the melting point of said second region.

31. The lipstick of claim 29 wherein said first region is opaque and second region is translucent or transparent.

32. The lipstick of claim 29 wherein at least one axial end of said first region is exposed.

33. The lipstick of claim 32 wherein said first region comprises a color component.

34. The lipstick of claim 32 wherein said first region comprises a topically-active ingredient.

35. In a lipstick of the core-sheath type comprising two different compositions disposed in a core-sheath relationship along a longitudinal axis thereof, the improvement wherein a composition disposed as a sheath is translucent or transparent.

36. The improvement of claim 35 wherein at least one composition is opaque.

37. The improvement of claim 35 wherein said composition disposed as a sheath is essentially free of waxes.

38. The improvement of claim 35 wherein said composition disposed as a sheath comprises an emollient.

39. The improvement of claim 35 wherein said composition disposed as a sheath comprises a polyamide resin.

40. A lipstick comprising a plurality of substantially coextensive regions extending lengthwise of said lipstick wherein:
(a) at least one of said regions is translucent or transparent;
(b) a first region extending lengthwise of said lipstick is surrounded by a second region extending lengthwise of said lipstick;
(c) at least one axial end of said first region is exposed; and
(d) said first region comprises a color component.
